(19) United States
(12) Patent Application Publication
(10) Pub. No.: US 2018/0153616 A1
(43) Pub. Date: Jun. 7, 2018

(54) METHODS OF TREATING CANCEROUS TISSUE

(71) Applicant: Wake Forest University Health Sciences, Winston-Salem, NC (US)

(72) Inventors: Suzy V. Torti, Avon, CT (US); Frank M. Torti, Avon, CT (US); Andrew Ryan Burke, Germantown, MD (US); Ravi Nandan-Singh, Gastonia, NC (US)

(21) Appl. No.: 15/815,158

(22) Filed: Nov. 16, 2017

Related U.S. Application Data
(63) Continuation of application No. 13/838,814, filed on Mar. 15, 2013, now abandoned.
(60) Provisional application No. 61/645,365, filed on May 10, 2012.

(51) Int. Cl. A61B 18/18; A61N 5/06; A61N 5/02

(52) U.S. Cl. CPC A61B 18/18; A61N 2005/0659; A61N 5/02; A61N 5/062

(57) ABSTRACT

In one aspect, methods of treating cancerous tissue are described herein. In some embodiments, a method of treating cancerous tissue comprises inducing necrotic cell death in cancer stem cells in vivo during hyperthermic treatment of the tissue, wherein inducing necrotic cell death comprises positioning nanoparticles adjacent to the cancer stem cells and irradiating the nanoparticles with electromagnetic radiation resulting in membrane damage to the cancer stem cells.

### CROSS REFERENCE TO RELATED APPLICATION

This application is a continuation application of U.S. patent application Ser. No. 13/838,814, filed Mar. 15, 2013, which claims priority pursuant to 35 U.S.C. § 119(e) to U.S. Provisional Patent Application Ser. No. 61/645,365, filed on May 10, 2012, each of which are hereby incorporated by reference in their entireties.

### STATEMENT REGARDING FEDERALLY SPONSORED RESEARCH

This invention was made with government support under grants R01CA128428, K99CA154006 and T32CA079448 awarded by the National Institutes of Health, and grant W81XWH-10-1-0332 awarded by the Department of Defense. The government has certain rights in the invention.

### FIELD

The present invention relates to methods of treating cancerous tissue and, in particular, to cancer stem cells.

### BACKGROUND

Many malignancies, including breast cancer, are thought to be sustained by a small, slow-cycling population of transformed stem-like cells that enable key aspects of disease progression, including expansion of the primary tumor and tumor metastasis. In addition, such cancer stem cells (CSCs) or tumor-initiating cells (TICs) are inherently refractory to standard treatments such as chemotherapy and radiotherapy. Moreover, CSCs are also resistant to traditional hyperthermic treatment. Failure to eliminate CSCs is believed to account for disease recurrence and/or metastasis in at least some instances.

### SUMMARY

In one aspect, methods of treating cancerous tissue are described herein. In some embodiments, a method of treating cancerous tissue comprises inducing cell death in cancer stem cells in vivo during hyperthermic treatment of the tissue, wherein inducing cell death comprises positioning nanoparticles adjacent to the cancer stem cells and irradiating the nanoparticles with electromagnetic radiation resulting in membrane damage to the cancer stem cells. Additionally, in some embodiments, the method further comprises inducing death in bulk cancer cells of the cancerous tissue, wherein the stem cells are not enriched in the remaining cell fraction subsequent to treatment. Further, in some embodiments of methods described herein, the proliferative ability of cancer stem cells not undergoing death subsequent to irradiation of the nanoparticles is diminished or abrogated.

In another aspect, a method of treating cancerous tissue comprises diminishing or abrogating the proliferative ability of cancer stem cells of the tissue in vivo, wherein diminishing or abrogating the proliferative ability comprises positioning nanoparticles in the cancerous tissue and irradiating the nanoparticles with electromagnetic radiation to heat the tissue, the cancer stem cells surviving the heating. Further, in some embodiments, bulk cancer cells are killed by the heating of the cancerous tissue.

These and other embodiments are described in greater detail in the detailed description which follows.

### BRIEF DESCRIPTION OF THE FIGURES

FIG. 1 is a series of graphs demonstrating the response of cancer stem cells and bulk cancer cells to hyperthermic therapy in the absence of nanoparticles.

FIG. 2 is a graph illustrating the percent increase in cancer stem cell fraction of a mixed population of cancer stem cells and bulk cancer cells following hyperthermic therapy in the absence of nanoparticles.

FIG. 3 is a series of graphs illustrating the viability of cancer cells following various treatments, including treatment according to some embodiments of methods described herein.

FIG. 4 is a graph demonstrating the percent change in cancer stem cell fraction of a mixed population of cancer stem cells and bulk cancer cells following various treatments, including treatment according to some embodiments of methods described herein.

FIG. 5 is a series of graphs displaying the viability of cancer cells following various treatments, including treatment according to some embodiments of methods described herein.

FIG. 6 is a series of graphs providing the 7-AAD positivity of cancer cells following various treatments, including treatment according to some embodiments of methods described herein.

FIG. 7 is a series of representative dot plots (flow cytometry) of cancer stem cells showing 7-AAD uptake and Annexin V labeling as a function of time following various treatments, including treatment according to some embodiments of methods described herein.

FIG. 8 is a series of graphs demonstrating median mammosphere diameters of cancer stem cells following various treatments, including treatment according to some embodiments of methods described herein.

FIG. 9 is a series of Kaplan-Meier plots showing the survival of tumor-bearing mice following various treatments, including treatment according to some embodiments of methods described herein.

### DETAILED DESCRIPTION

Embodiments described herein can be understood more readily by reference to the following detailed description, examples, and drawings. Elements, apparatus, and methods described herein, however, are not limited to the specific embodiments presented in the detailed description, examples and drawings. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

Further, when the phrase “up to” is used in connection with an amount, it is to be understood that the amount is at least a detectable amount. For example, a material present in an amount “up to” a specified amount can be present from a detectable amount and up to and including the specified amount.

In one aspect, methods of treating cancerous tissue are described herein. In some embodiments, a method of treating cancerous tissue comprises inducing cell death in cancer stem cells in vivo during hyperthermic treatment of the tissue, wherein inducing cell death comprises positioning nanoparticles adjacent to the cancer stem cells and irradiating the nanoparticles with electromagnetic radiation resulting in membrane damage to the cancer stem cells. The cancerous tissue, in some embodiments, comprises breast cancer tissue, and the cancer stem cells comprise breast cancer stem cells. In some embodiments, the cancerous tissue and the cancer stem cells over-express the oncogene RAS. In some embodiments, the cancer stem cells demonstrate a triple negative phenotype by lacking expression of estrogen receptors, progesterone receptors and HER-2. Additionally, in some embodiments, the cancer stem cells display a CD44^{high}/CD24^{low} antigen profile.

Cancer stem cells treated with methods described herein, in some embodiments, are associated with one or more cancers selected from the group consisting of gastrointestinal cancer, lung cancer, colon cancer, skin cancer, melanoma, brain cancer, prostate cancer, testicular cancer, ovarian cancer, liver cancer, leukemia, glioblastoma, head and neck cancer, bladder cancer, myeloma and pancreatic cancer.

In some embodiments, stem cells of the cancerous tissue overexpress one or more heat shock proteins (HSPs). In some embodiments, for example, stem cells of the cancerous tissue overexpress HSP90. Moreover, in some embodiments, stem cells of the cancerous tissue overexpress one or more HSPs by about 2-fold to about 10-fold compared to bulk cancer cells of the tissue. In some embodiments, stems cells of the treated tissue are resistant to chemotherapeutics such as doxorubicin or other thermal therapies.

In some embodiments of methods described herein, cell death is induced in at least 50% of the cancer stem cells of the tissue. Cell death, in some embodiments, is induced in at least 60% or at least 70% of the cancer stem cells. In some embodiments, cell death is induced in at least 80% or at least 90% of the cancer stem cells. In some embodiments, cell death is induced in at least 95% or at least 99% of the cancer stem cells of the tissue. In some embodiments, cell death is solely necrotic cell death or solely apoptotic cell death. Cell death, in some embodiments, is induced by a combination of necrotic and apoptotic mechanisms. In some embodiments, for example, a portion of the cancer stems cells undergo necrotic cell death and another portion of the stem cells undergo apoptotic cell death.

In some embodiments wherein necrotic cell death is induced in cancer stem cells, methods described herein can bypass one or more apoptotic death mechanisms that permit cancer stem cells to resist treatments lacking nanoparticle mediation, including traditional hyperthermic techniques as well as various chemotherapy and radiotherapy treatment methods.

Cell death, according to some embodiments of methods described herein, is caused by membrane damage to the cancer stem cells. Membrane damage, in some embodiments, comprises membrane permeabilization, including irreversible membrane permeabilization. In some embodiments, for example, more than about 50% of the cancer stem cells in the tissue exhibit membrane permeabilization. In some embodiments, more than about 60% or more than about 70% of the cancer stem cells exhibit membrane permeabilization. In some embodiments, more than about 80% or more than about 90% of the cancer stem cells exhibit membrane permeabilization. As provided in the Examples herein, membrane permeabilization, in some embodiments, can be determined by 7-AAD positivity of a cell population.

In some embodiments of methods described herein, not all cancer stem cells in a cancerous tissue undergo cell death in response to nanoparticle irradiation. Nevertheless, the proliferative ability of the surviving cancer stem cells subsequent to irradiation of the nanoparticles is diminished or abrogated. The proliferative ability of cancer stem cells, in some embodiments, comprises the ability to propagate as floating spheroids, such as tumorspheres, in non-adherent conditions. In some embodiments, at least 95% of cancer stem cells surviving subsequent to nanoparticle irradiation lack proliferative ability. In some embodiments, at least 99% of surviving cancer stem cells lack proliferative ability. In some embodiments, all or substantially all of surviving cancer stem cells lack proliferative capacity. The ability of methods described herein to diminish or abrogate proliferative ability of the cancer stem cells, in some embodiments, precludes recurrence, redevelopment and/or metastasis of the cancerous tissue.

Moreover, in some embodiments, a method described herein further comprises inducing death in bulk cancer cells of the cancerous tissue. In some embodiments, the death of the bulk cancer cells is necrotic cell death, apoptotic cell death or a combination thereof. In some embodiments, the death of the bulk cancer cells is primarily necrotic cell death. Death of bulk cancer cells, in some embodiments, does not lead to the enrichment of cancer stem cells in the remaining cell fraction after irradiation of the nanoparticles. In some embodiments, for instance, the cancer stem cells and bulk cancer cells exhibit equivalent or substantially equivalent sensitivity to a method of treating cancerous tissue described herein. In some embodiments, the cancer stem cells exhibit enhanced sensitivity.

Turning now to specific steps, methods of treating cancerous tissue described herein comprise positioning nanoparticles in cancerous tissue adjacent to cancer stem cells. Any nanoparticles not inconsistent with the objectives of the present invention may be used. In some embodiments, for instance, the nanoparticles have an aspect ratio greater than 1. In some embodiments, the nanoparticles have an aspect ratio ranging from about 1.1 to about 10,000. In some embodiments, the nanoparticles have an aspect ratio ranging from about 10 to about 1,000 or from about 10 to about 100. The nanoparticles, in some embodiments, have an aspect ratio ranging from about 5 to about 50.

In some embodiments, nanoparticles of a method described herein have a length ranging from about 10 nm to about 3 μm or from about 50 nm to about 2 μm. In some embodiments, the nanoparticles have a length ranging from about 100 nm to about 1.5 or from about 500 nm to about 1 μm. In some embodiments, the nanoparticles have a length ranging from about 300 nm to about 700 nm or from about 400 nm to about 600 nm. In some embodiments, the nanoparticles have a length greater than about 1 μm or a length ranging from about 1 μm to about 3 μm. In some embodiments, the nanoparticles have a length greater than 3 μm.

Additionally, in some embodiments, the nanoparticles have a diameter less than about 200 nm. In some embodiments, the nanoparticles have a diameter ranging from about 5 nm to about 150 nm or from about 10 nm to about 100 nm. In some embodiments, the nanoparticles have a diameter ranging from about 10 nm to about 50 nm.

Nanoparticles of methods described herein, in some embodiments, comprise organic nanoparticles, inorganic nanoparticles or mixtures thereof. Organic nanoparticles, in some embodiments, comprise carbon nanoparticles. In some embodiments, carbon nanoparticles comprise carbon nanotubes, including single-walled carbon nanotubes (SWNT) and/or multi-walled carbon nanotubes (MWNT). Carbon nanotubes, in some embodiments, have branched structures. Branched structures, in some embodiments, comprise multiple branches, Y branches, Y branches with multiple branches and multi-level Y branches.

Carbon nanotubes, in some embodiments, are doped with boron, nitrogen or combinations thereof. In some embodiments, for example, doped carbon nanotubes comprise boron in an amount ranging from about 0.01 weight percent to about 10 weight percent. In some embodiments, doped carbon nanotubes comprise boron in an amount ranging from about 1 weight percent to about 5 weight percent. In some embodiments, doped carbon nanotubes comprise nitrogen in an amount ranging from about 0.01 weight percent to about 30 weight percent or from about 5 weight percent to about 25 weight percent. In some embodiments, doped carbon nanotubes comprise nitrogen in an amount greater than about 30 weight percent. In some embodiments, doped carbon nanotubes comprise nitrogen in an amount ranging from about 10 weight percent to about 20 weight percent. In some embodiments, doped carbon nanotubes comprise less than about 1 weight percent nitrogen.

Further, in some embodiments, carbon nanotubes comprise one or more transition metals, including iron, cobalt, nickel, silver or combinations thereof. In some embodiments, a carbon nanotube comprises at least about 0.01 weight percent of a transition metal. In some embodiments, a carbon nanotube comprises a transition metal in an amount ranging from about 0.5 weight percent to about 3 weight percent or from about 1 weight percent to about 2 weight percent. In some embodiments, a transition metal is disposed in the cavity of a nanotube or between walls of a MWNT. In some embodiments, a transition metal is attached to a surface of a nanotube or incorporated into the lattice of the nanotube.

In some embodiments, carbon nanotubes comprise at least one positive magnetic resonance (T1) contrast agent. In some embodiments, for example, a positive contrast agent includes chemical species comprising gadolinium, such as gadolinium chloride. In some embodiments, the magnetic resonance contrast agent is disposed within the nanotube. The magnetic resonance contrast agent, in some embodiments, is disposed on a surface of the carbon nanotube. In some embodiments, carbon nanotubes comprising iron and/or a positive contrast agent are doped with nitrogen and/or boron.

In some embodiments, the nanoparticles comprise graphene, nanohorns, fullerite, fullerene or mixtures thereof.

Alternatively, in some embodiments, nanoparticles of a method described herein comprise inorganic nanoparticles. In some embodiments, inorganic nanoparticles comprise nanoshells, nanorods, nanowires, nanotubes, or mixtures thereof. Inorganic nanoparticles, in some embodiments, comprise metals, including transition metals, noble metals, alkali metals and alkaline-earth metals. Inorganic nanoparticles, in some embodiments, comprise metal oxides such as zinc oxide, titanium oxide or combinations thereof. In some embodiments, inorganic nanoparticles comprise boron nitride. In some embodiments, inorganic nanoparticles comprise semiconductor materials, including II/VI and III/V semiconductors. In some embodiments, inorganic nanoparticles are nanotubes or nanorods. In some embodiments, an inorganic nanotube or nanorod comprises one or more of zinc oxide, titanium oxide and boron nitride.

In some embodiments, nanoparticles for use in methods described herein, including carbon nanotubes, are surface functionalized with one or more hydrophilic chemical species. Hydrophilic chemical species suitable for functionalizing nanoparticle surfaces, in some embodiments, comprise species having carboxyl, sulfonic, amine and/or amide functionalities. In some embodiments, suitable hydrophilic chemical species can comprise hydrophilic polymers including, but not limited to, poly(dimethyldiallylammonium chloride), polyethylene glycol, alkoxylated polyethylene glycol or polypropylene glycol.

In some embodiments, nanoparticles described herein are functionalized by covalently linking a hydrophilic chemical species to the surface. In some embodiments, nanoparticle surfaces are functionalized by forming non-covalent intermolecular interactions with a hydrophilic chemical species, including ionic, dipole-dipole, electrostatic and/or van der Waals interactions. In a further embodiment, surfaces of nanoparticles are functionalized by forming covalent and non-covalent interactions with one or more hydrophilic chemical species. Functionalization of nanoparticle surfaces with hydrophilic chemical species can increase the solubility and/or dispersion of the nanoparticles in polar media.

In some embodiments, nanoparticles described herein, including carbon nanotubes, are disposed in a physiologically acceptable carrier in a therapeutically effective amount for introduction in the cancerous tissue. A therapeutically effective amount of nanoparticles can depend on several factors including identity of the nanoparticles, volume of the cancerous tissue to be treated and the type of cancerous tissue to be treated. In some embodiments, nanoparticles described herein are disposed in a physiologically acceptable carrier at a concentration ranging from about 0.1 μg/ml to about 5 mg/ml. Nanoparticles, in some embodiments, are disposed in a physiologically acceptable carrier at a concentration ranging from about 1 μg/ml to about 2 mg/ml, from about 10 μg/ml to about 500 μg/ml, from about 25 μg/ml to about 250 μg/ml or from about 30 μg/ml to about 100 μg/ml. In a further embodiment, nanoparticles are disposed in a physiologically acceptable carrier at a concentration greater than about 1 mg/ml or less than about 0.1 μg/ml. In one embodiment, nanoparticles are disposed in a physiological carrier at a concentration ranging from about 40 μg/ml to about 60 μg/ml.

Physiologically acceptable carriers, according to some embodiments, comprise solutions or gels compatible with human and/or animal tissue. In some embodiments, physiologically acceptable carriers comprise water, saline solutions and/or buffer solutions. Buffer solutions, in some embodiments, comprise carbonates, phosphates (e.g., phosphate buffered saline), acetates or organic buffers such as tris(hydroxymethyl)aminoethane (Tris), N-2-hydroxyethylpiperazine-N′-2-ethanesulfonic acid (HEPES) or 3-(N-morpholino)propanesulfonic acid (MOPS). In some embodiments, gels comprise hydrogels, such as those constructed from polyvinyl alcohol, or dextran such as carboxymethyl dextran.

In some embodiments, a physiologically acceptable carrier comprises ethylene oxide and propylene oxide copolymers such as those available from BASF of Florham Park, N.J. under the tradename PLURONIC®. A physiologically acceptable carrier, in some embodiments, comprises collagen, chitosan, alginates or combinations thereof. Moreover, physiologically acceptable carriers, in some embodiments, comprise dispersants such as poly(diallyldimethylammonium chloride) (PDDA), surfactants or combinations thereof. In a further embodiment, a physiologically acceptable carrier comprises poly(lactic)-co-glycolic acid, fibrinogin, chondroitan or combinations thereof.

As described herein, the nanoparticles are positioned adjacent cancer stem cells in the tissue. Positioning the nanoparticles adjacent to cancer stem cells can be carried out in any manner not inconsistent with the objectives of the present invention. For example, in some embodiments, positioning nanoparticles comprises injecting the nanoparticles disposed in a physiologically acceptable carrier into the cancerous tissue, including through one or more local injections. In some embodiments, positioning nanoparticles comprises injecting the nanoparticles percutaneously into the cancerous tissue. In some embodiments, positioning the nanoparticles further comprises allowing the nanoparticles to diffuse within the cancerous tissue for a desired period of time. In some embodiments, the nanoparticles are allowed to diffuse in the cancerous tissue for a time period of 1 hour, 4 hours, 6 hours, 12 hours or 24 hours. In some embodiments, the nanoparticles are allowed to diffuse within the cancerous tissue for a time period of less than 1 hour or greater than 24 hours.

Moreover, in some embodiments described herein, the nanoparticles contact cancer stem cells. In some embodiments, the nanoparticles are in contact with cell membranes of the cancer stem cells. In one embodiment, for example, nanoparticles are partially imbedded in cell membranes of the cancer stem cells. While being in contact with cancer stem cells, nanoparticles, in some embodiments, do not enter the stem cells and remain in the extracellular environment. Additionally, in some embodiments, the nanoparticles remain at least partially outside the cancer stem cells throughout the hyperthermic treatment of the tissue.

Alternatively, in some embodiments, the nanoparticles are not in direct contact with cancer stem cells. In some embodiments, the nanoparticles are positioned within a subcellular distance from cancer stem cells. For example, in some embodiments, the nanoparticles are less than about 100 μm or less than about 10 μm away from cancer stem cells. In some embodiments, the nanoparticles are between about 1 μm and about 10 μm away from cancer stem cells. In some embodiments, the nanoparticles are less than about 1 μm away from cancer stem cells. In some embodiments, the nanoparticles are between about 1 nm and about 1000 nm or between about 1 nm and about 100 nm away from cancer stem cells. In some embodiments, the nanoparticles are between about 1 nm and about 10 nm, between about 50 nm and about 500 nm or between about 100 nm and about 1000 nm away from cancer stem cells.

Once positioned adjacent to the cancer stem cells, the nanoparticles are irradiated with electromagnetic radiation from a radiation source. In some embodiments, the nanoparticles are irradiated with infrared radiation. In some embodiments, infrared radiation comprises near infrared radiation (NIR), mid-wavelength infrared radiation (MWIR), long-wavelength infrared radiation (LWIR) or combinations thereof. In some embodiments, for example the nanoparticles are irradiated with radiation having a wavelength between about 700 nm and about 1100 nm, between about 1000 nm and about 1100 nm or between about 1250 nm and about 1350 nm. In some embodiments, the nanoparticles are irradiated with microwave or radio frequency radiation. The nanoparticles, in some embodiments, are irradiated with radiation having a wavelength between about 3 μm and about 5 μm, between about 10 μm and about 12 μm or between about 1 cm and about 15 cm.

The source of electromagnetic radiation, in some embodiments, is external to a patient's body. Alternatively, the source of electromagnetic radiation, in some embodiments, is at least partially within a patient's body during administration of methods described herein. For example, in some embodiments wherein cancerous tissue is located at a depth sufficient to interfere with external administration of the radiation, fiber optics or similar devices can be used endoscopically to penetrate surrounding tissue and deliver radiation to nanoparticles.

A radiation source, in some embodiments, comprises a laser producing the desired wavelength(s) of radiation. In some embodiments, for example, a Nd:YAG laser is used for infrared irradiation of carbon nanotubes at one or more wavelengths. In some embodiments, a radiation source comprises a radio frequency probe.

In another aspect, a method of treating cancerous tissue described herein comprises diminishing or abrogating the proliferative ability of cancer stem cells of the tissue in vivo, wherein diminishing or abrogating the proliferative ability comprises positioning nanoparticles in the tissue and irradiating the nanoparticles with electromagnetic radiation to heat the tissue, the cancer stem cells surviving the heating. In some embodiments, at least 95% of cancer stem cells surviving subsequent to nanoparticle irradiation lack proliferative ability. In some embodiments, at least 99% of surviving cancer stem cells lack proliferative ability. In some embodiments, all or substantially all of surviving cancer stem cells lack proliferative capacity. The ability of methods described herein to diminish or abrogate proliferative ability of the cancer stem cells, in some embodiments, precludes recurrence, redevelopment and/or metastasis of the cancerous tissue.

Further, in some embodiments, bulk cancer cells are killed in the heating of the cancerous tissue. The bulk cancer cells, in some embodiments, display necrotic death, apoptotic death or a combination thereof. Additionally, in some embodiments, a portion of cancer stem cells are killed in the heating of the cancerous tissue, wherein the cancer stem cells surviving the heating demonstrate diminished or abrogated proliferative ability according to embodiments described herein.

Nanoparticles and sources of radiation suitable for use in the present method can comprise any of the same recited herein.

Some embodiments described herein are further illustrated in the following non-limiting examples.

### Example 1

### Cancer Cells

Model cancer cells used in the following Examples were prepared as follows. First, breast cancer stem cells (HMLER^{shEcadherin}) and bulk (non-stem) breast cancer cells (HMLER^{shControl}) were obtained from the laboratory of Dr. Robert Weinberg (MIT). The model cancer stem cells exhibited a mesenchymal morphology; a 20-fold increase in cells displaying the CD44^{high}/CD24^{low} antigen profile characteristic of tumor initiating breast cancer stem cells; the ability to propagate as floating spheroids (e.g., mammospheres or tumorspheres) in non-adherent conditions; and a 10-fold increase in resistance to the chemotherapeutic drug paclitaxel. The model cancer stem cells also displayed properties of triple negative breast cancer cells, lacking expression of estrogen receptors, progesterone receptors, and HER-2. The cancer stem cells and bulk cancer cells were cultured in a 1:1 mixture of mammary epithelial cell growth medium (MEGM) and Dulbecco's modified Eagle's medium (DMEM) supplemented with 10% fetal bovine serum (FBS), insulin, and hydrocortisone in humidified incubators maintained at 37° C. with 5% CO₂.

### Example 2

### Carbon Nanotubes

Suspensions of carbon nanotubes suitable for use in some embodiments of methods of treating cancerous tissue described herein were prepared as follows. First, amide-functionalized multi-walled carbon nanotubes (MWCTs, PD15L1-5-NH₂, lot number 60809) were purchased from NanoLab (Waltham, Mass.). The MWCTs were then suspended in sterile saline with 1% (wt/wt) DSPE-PEG 5000 (Avanti Polar Lipids) through probe tip sonication (Branson). All preparations were autoclaved prior to use. Physico-chemical characterization of this material is provided in Burke et al., “Determinants of the thrombogenic potential of multiwalled carbon nanotubes,” Biomaterials 2011, 32, 5970-5978.

### Example 3

### Viability of Cancer Cells

The viability of cancer cells treated according to some embodiments of methods described herein was investigated as follows. First, the sensitivity of the cancer stem cells and bulk cancer cells of Example 1 to traditional hyperthermic treatment was evaluated. The cancer stem cells and bulk cancer cells were heated to a temperature between 43° C. and 49° C. in a circulating water bath, and changes in cell viability over time were determined. In particular, cells were resuspended in normal growth media (250,000 cells in 500 μL media) and placed in microcentrifuge tubes. Triplicate samples were prepared for each cell type. Samples were then placed in a circulating water bath set at the desired temperature. The tubes were removed from the water bath at the indicated time points, and 100 μL volumes were withdrawn from each sample and plated in a 96-well plate. Next, 100 μL of fresh media was added to each well and plates were allowed to recover overnight at 37° C. Wells were then prepared for viability analysis as described below.

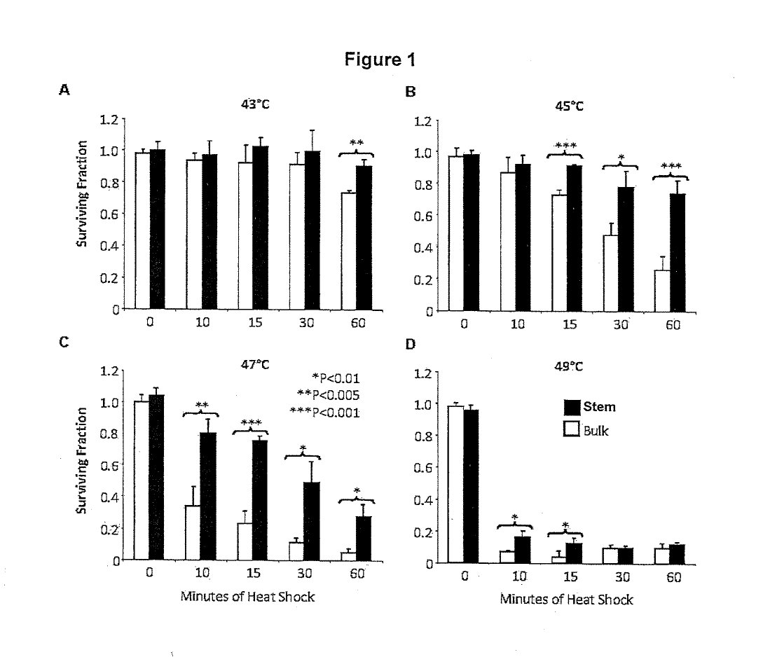

As shown in FIG. 1, cancer stem cells were significantly more resistant to the effects of hyperthermia than bulk cancer cells across the entire temperature range. For example, at a treatment temperature of 47° C., viability of the bulk cancer cells (expressed as a fraction relative to untreated cells) following 10, 15, 30, and 60 minutes of treatment was reduced to 0.33, 0.23, 0.11, and 0.05. In contrast, hyperthermic treatment under identical conditions reduced the viability of the cancer stem cells to 0.81, 0.76, 0.49, and 0.28. Thus, the thermal resistance of cancer stem cells compared to bulk cancer cells at 47° C. ranged from 2.7-fold to 5.6-fold. In FIG. 1, P-values correspond to the indicated pair-wise comparisons and were determined by post-hoc Student's t-test following an ANOVA that determined overall significance. All statistical analyses were performed with SPSS software.

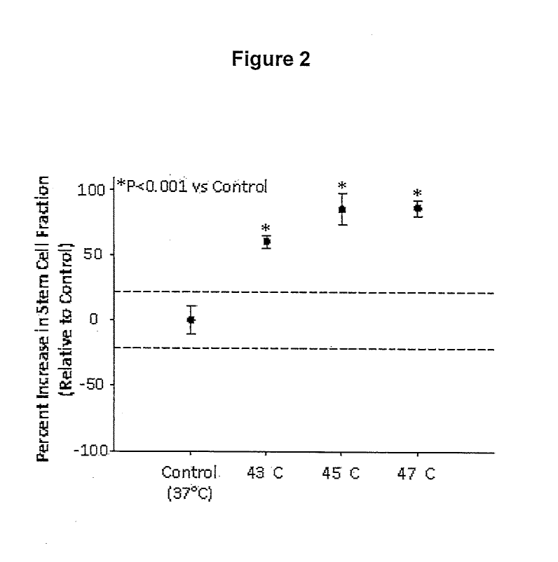

Further, a population of cells including both cancer stem cells and bulk cancer cells became enriched in cancer stem cells after hyperthermic treatment in a circulating water bath at 43° C., 45° C., and 47° C. for 75, 30, and 5 minutes, respectively. The hyperthermic treatment killed 67.7%, 71.3%, and 82% of the starting cells, respectively. Surviving cells were analyzed by flow cytometry to determine the relative percentage of CD44^{high}/CD24^{low} cells. The cancer stem cell fraction increased from 15.7±1.7% before treatment to 25.3±0.7% (43° C.), 29.2±1.8% (45° C.) and 29.2±0.9% (47° C.) following treatment (24 hours post-treatment). FIG. 2 shows the percent increase in the cancer stem cell fraction. The dashed lines indicate the 95% confidence interval (C.I.) for the untreated cells (labeled “Control”). Significance was determined by ANOVA with post-hoc Student's t-tests. For flow cytometry analysis, cells were washed and resuspended at 1×10⁶ cells in 100 μL FACS buffer. Cells were labeled at 4° C. for 30 minutes with APC mouse anti-human CD24 (ML5, Biolegend), PerCP-Cy5.5 mouse anti-human CD44 (C26, BD Pharmingen), both or the appropriate isotypes (APC mouse IgG2a κ isotype (Biolegend) and PerCP-Cy5.5 mouse IgG2b κ isotype (BD Pharmingen)) at the manufacturer's recommended concentrations. Samples were then washed once with cold phosphate buffered saline (PBS) and fixed with 1× formaldehyde in PBS. Cells were analyzed on a FACS Aria (Becton Dickinson) or an Accuri C6. Data was exported and graphed using FCS Express (DeNovo Software).

Next, populations of cancer cells were treated according to one embodiment of a method described herein. A series of cell suspensions described in Example 1 were mixed with the MWCTs of Example 2 to generate a final MWCT concentration of 50 μg/mL in 500 μL total sample volume. Baseline temperature measurements of each cell sample were then acquired by thermocouple and used to determine the change in temperature necessary to reach a desired final temperature between 43° C. and 49° C. The cells were then immediately exposed to 3-W NIR laser light for lengths of time calculated to generate the desired final temperatures. The required exposure time to reach a given final temperature was determined according to the following heating model: T=0.627t+1.6971, where T is the desired change in temperature in degrees Celsius and t is the laser exposure time in seconds. Exposure times ranged from 5 to 46 seconds. As controls, samples that included cells mixed with MWCTs but did not receive laser irradiation (termed “CNT Only” samples) and samples that included cells not mixed with MWCTs but that did receive laser irradiation (termed “Laser Only” samples) were also generated. Immediately following laser irradiation, cells were washed extensively to remove MWNTs and then replated and allowed to recover overnight.

Viability of the cell samples was assessed by MTT as follows. Cells were dispensed in 96-well plates. Wells were washed with 200 μL PBS and overlaid with 200 μL fresh media prior to initiating the assay. Then 50 μL of MTT reagent (5 mg/mL thiazolyl blue tetrazolium bromide in PBS, Sigma) was added to each well, followed by incubation at 37° C. for 1-2 hours. Three wells that did not contain cells were similarly treated and used as blanks. After incubation, media was aspirated and formazan crystals were solubilized in 200 μL dimethyl sulfoxide (DMSO). The pH was adjusted by adding 25 μL Sorensen glycine buffer (pH 10.5) per well. Well contents were then mixed for 10 minutes on a titer plate shaker. Absorbance at 560 nm was determined using a plate reader (Molecular Devices). Absorbance at 490 nm for each treatment group was averaged then normalized to the indicated control conditions.

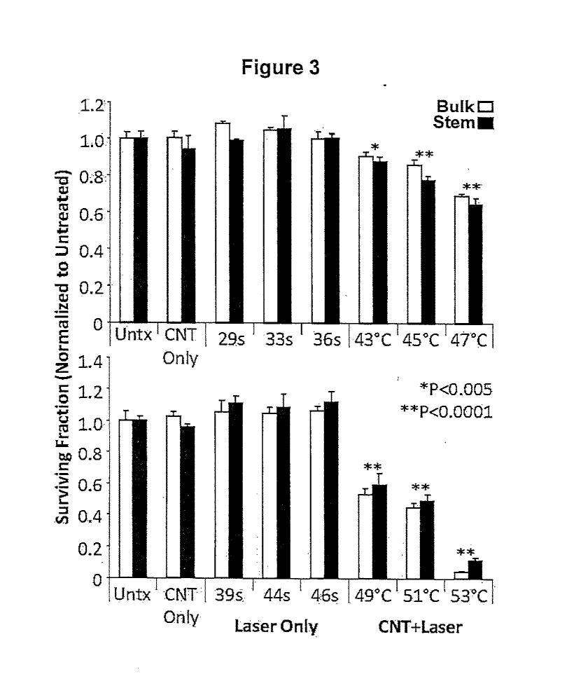

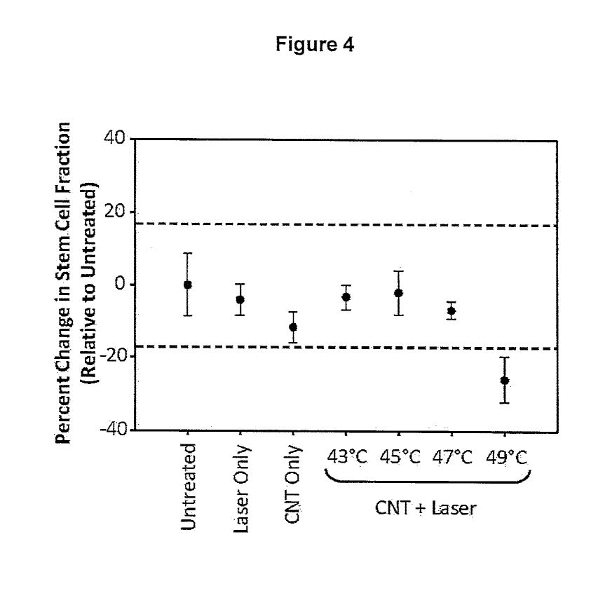

As indicated in FIG. 3, the cancer stem cells were very sensitive to nanotube-mediated thermal therapy (NMTT). In FIG. 3, viability (MTT) results for NMTT (labeled “CNT+Laser”) are compared to results for no treatment (“Untx”), treatment with carbon nanotubes only (“CNT Only”), and treatment with laser irradiation only (“Laser Only”). P-values indicate significant differences for both cell types relative to the untreated conditions. Statistical significance was determined by ANOVA and post-hoc Student t-tests. The sensitivity of cancer stem cells to NMTT contrasts with the water bath hyperthermic treatment results. Moreover, to confirm that cancer stem cells and bulk cancer cells had substantially equivalent sensitivity to NMTT, the surviving fraction was allowed to recover for 24 hours and then analyzed by flow cytometry to determine the CD44^{high}/CD24^{low} cancer stem cell fraction. The results are shown in FIG. 4, normalized to the untreated condition (defined as zero percent change). Dashed lines indicate the 95% C.I. for the untreated condition. Significance was determined by ANOVA with post-hoc Student's t-tests. As illustrated in FIG. 4, the cancer stem cells were not enriched in the remaining cell fraction.

To verify that the effectiveness of NMTT for inducing cell death in cancer cells was not due only to the rate of heating achieved with NMTT, the water bath hyperthermic treatment was modified to produce a more rapid rate of temperature increase (ROTI). Prior to modification, the water bath hyperthermic treatment produced a ROTI calculated to be 0.1-0.2° C./second (“slow ROTI”). The ROTI achieved during NMTT was 0.6° C./second. To achieve a “rapid ROTI,” the slow ROTI water bath hyperthermic treatment described above was modified as follows. First, 10 mL of normal growth media in 15 mL conical tubes was preheated to 43-53° C. by immersion in a temperature controlled circulating water bath. Next, 250,000 cells were suspended in 100 μL normal growth media and rapidly injected into the preheated media. Cells were maintained at the preset temperatures for 30 seconds, followed by cooling on ice. The rapid ROTI water bath hyperthermic treatment process produced a calculated temperature increase of 5000° C./second. Cancer stem cells were treated to reach 43-53° C. by either the “slow” or “rapid” water bath hyperthermic treatment.

Following heat treatment, samples were centrifuged and cell pellets resuspended in 500 μL growth media. Then 100 μL volumes were withdrawn from each sample and plated in a 96-well plate. Next, 100 μL of fresh media was added to each well, and plates were allowed to recover overnight at 37° C. Wells were then prepared for viability analysis as described above.

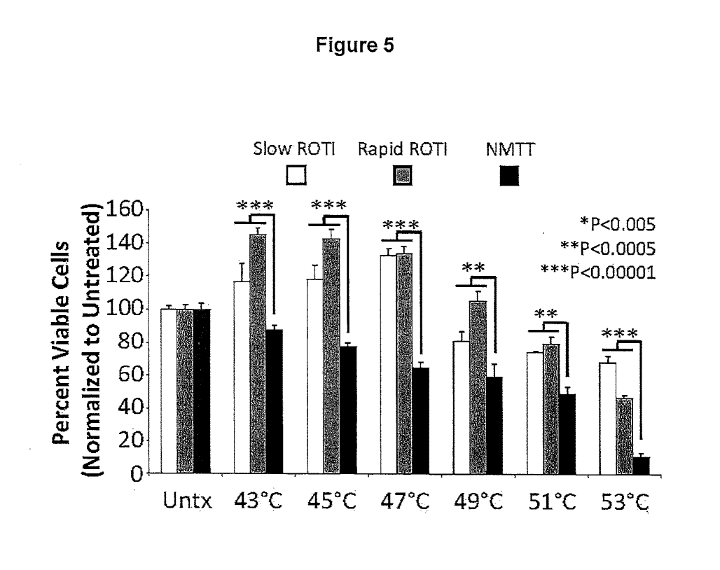

As shown in FIG. 5, a rapid ROTI did not enhance cell death. Moreover, NMTT was significantly more cytotoxic to cancer stem cells than either water bath hyperthermic treatment. For example, in cells treated to reach 43-49° C., rapid ROTI hyperthermic treatment increased cell counts at 24 hours post-treatment to 128.8%, 146.9%, 136.2% and 110.8% of untreated (“Untx”), respectively. In contrast, NMTT at the same time and treatment temperatures decreased cell counts to 87.6%, 77.7%, 64.8% and 59.4% of untreated. In FIG. 5, P-values correspond to the statistical differences between NMTT and water bath hyperthermic treatments at a given temperature. Overall significance was determined by ANOVA followed by post-hoc Student's t-tests for the pair-wise comparisons.

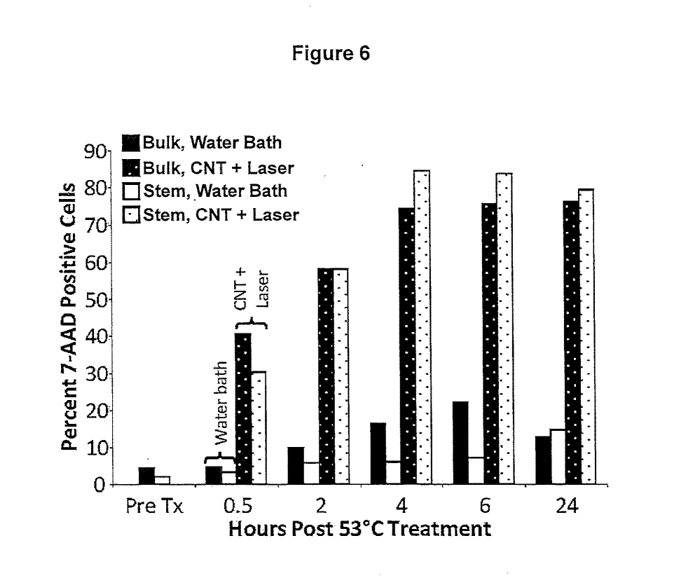

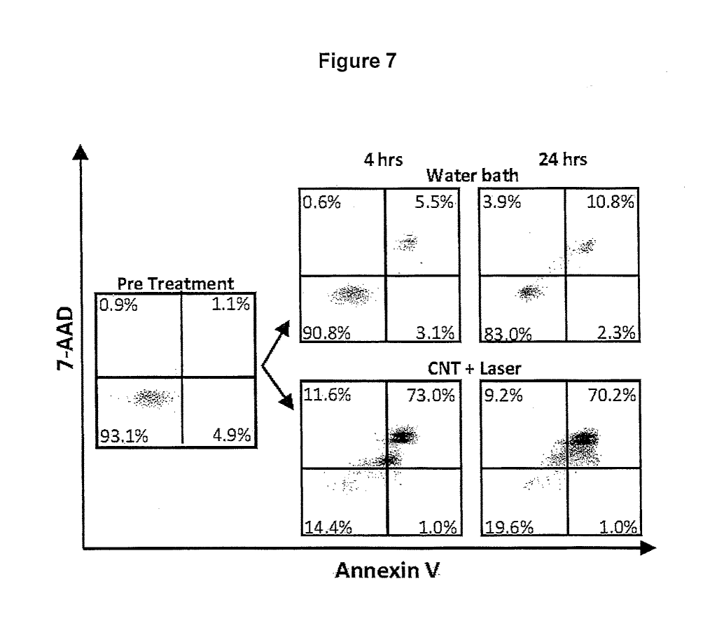

To determine the mechanism of cell death induced by NMTT, cancer stem cells and bulk cancer cells were treated with NMTT to a final temperature of 53° C., followed by measurement of Annexin V labeling (a marker of apoptosis) and 7-AAD permeability (an indicator of plasma membrane integrity) at time points ranging from 30 minutes to 24 hours post treatment. As a control, cells were also treated with water bath hyperthermic treatment with a rapid ROTI. As indicated in FIG. 6, 7-AAD positivity for water bath treated cells (“Water Bath”) increased for cancer stem cells and bulk cancer cells, respectively, from 2.1% and 4.6% pre-treatment (“Pre Tx”) to 14.7% and 12.8% at 24 hours post-treatment. For cells treated with NMTT (“CNT+Laser”), 7-AAD positivity reached 76.3% and 79.4%, respectively, for cancer stem cells and bulk cancer cells over the same time period. The 7-AAD uptake served as a quantitative marker for both cell death and also membrane permeabilization. As indicated in FIG. 6, a majority of cancer stem cells exhibited membrane permeabilization following NMTT. In addition, cell death occurred more rapidly following NMTT than water bath hyperthermic treatment. Moreover, sustained increases in apoptotic cells were not seen over the 24 hours of the study, as shown in FIG. 7, indicating that necrosis was the primary form of cell death in both cancer stem cells and bulk cancer cells following NMTT. FIG. 7 is a series of representative dot plots of cancer stem cells showing 7-AAD uptake and Annexin V labeling as a function of time following heat treatment.

### Example 4

### Proliferative Ability of Cancer Cells

The proliferative ability of cancer cells treated according to some embodiments of methods described herein was investigated as follows. The cancer stem cells of Example 1 were suspended at 100,000 cells per 100 μL normal growth media. Cell suspensions were then heat treated to 43° C., 45° C., 47° C. or 49° C. by either rapid ROTI water bath hyperthermic treatment or NMTT as described in Example 3. Immediately after treatment, the cell suspensions were centrifuged and washed twice with PBS. The cells were then resuspended at 5,000 cells/mL in complete Mammocult media (Stem Cell Technologies) and plated in triplicate wells of a 6-well ultra-low attachment culture plate (Corning) as single-cell suspensions to track mammosphere formation. Cells were incubated at 37° C. for 7-10 days. To form a mammosphere of about 200 μm in diameter, a single cell must undergo approximately 10 rounds of replication. Wells were imaged by inverted microscope (Olympus IX70) and mammosphere diameters were determined using the Image J software package. At least 50 cells or cell clusters (mammospheres) were counted per condition. Single cells had diameters of about 15-25 μm.

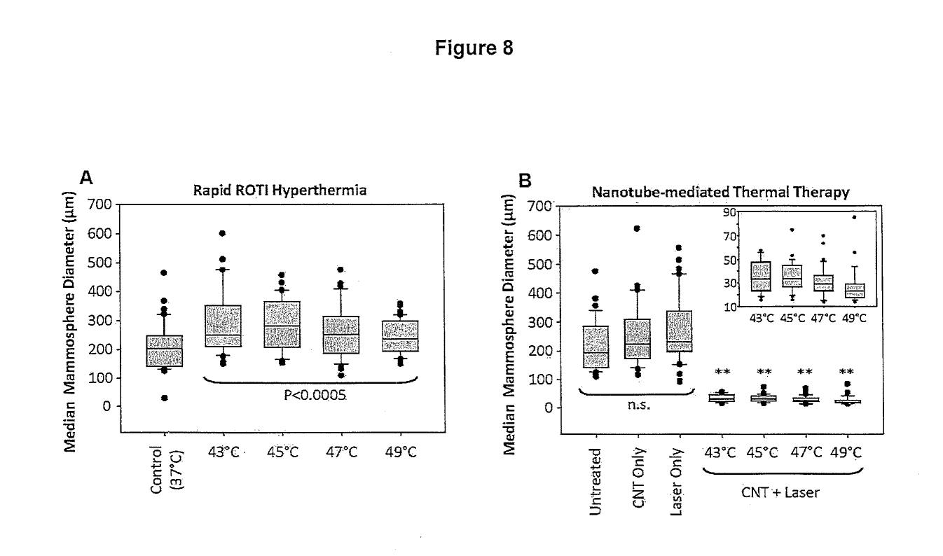

As shown in FIG. 8, rapid ROTI water bath hyperthermic treatment led to significantly increased mammosphere size. Specifically, by day 7, median mammosphere size increased from 203.8 μm in the untreated condition (“Control”) to 250 μm, 281.6 μm, 250.8 μm and 236.3 μm in the 43° C., 45° C., 47° C. and 49° C. water bath hyperthermic treatment groups, respectively (P=0.00028) (FIG. 8A). These findings are consistent with data shown in FIG. 5 indicating that rapid ROTI water bath hyperthermic treatment promotes increased cell proliferation as early as 24 hours following treatment. In contrast, NMTT completely abrogated the mammosphere-forming ability of cancer stem cells (FIG. 8B). FIG. 8 shows median mammosphere diameters for each treatment group along with 25^{th} and 75^{th} percentiles. Outlier values are indicated by filled circles. At least 40 mammospheres were measured per treatment group. The inset of FIG. 8B provides detail on median mammosphere sizes formed after NMTT. Statistical differences were determined by ANOVA. Double asterisks (**) indicate P<0.001 relative to untreated cells.

### Example 5

### In Vivo Study

A method of treating cancerous tissue according to one embodiment described herein was carried out as follows. All animal studies were performed in compliance with the institutional guidelines on animal use and welfare (Animal Care and Use Committee of Wake Forest University Health Sciences) under an approved protocol. Female nu/nu athymic mice were obtained from Charles River Laboratories (5-8 weeks old). Mice were housed 5 per cage in standard plastic cages, provided food and water ad libitum, and maintained on a 12-hour light/dark cycle.

To determine the efficacy of NMTT in vivo, athymic mice were implanted subcutaneously with cancer stem cells of Example 1. Specifically, one athymic female mouse was injected subcutaneously with 2×10⁶ cancer stem cells suspended in 100 of 1:1 Matrigel (BD Biosciences) and PBS. When the tumor reached a size of about 1,000 mm³ it was resected and minced under sterile conditions into 30 mm³ fragments. Fragments were surgically implanted into the flanks of 50 athymic female mice and allowed to grow to about 150 mm³ over the course of 7-10 days.

Mice were then randomized into 3 control (Untreated, Laser Only and CNT Only) and one experimental group (CNT+Laser) with 10 animals per group. Mice in the “Laser Only” group received an intratumoral injection of 50 μL saline with 1% DSPE-PEG. Mice in the “CNT Only” and “CNT+Laser” groups received intratumoral injections of 100 μg of the MWNTs of Example 2 suspended in sterile saline with 1% DSPE-PEG. Following injection, mice in the “Laser Only” and “CNT+Laser” groups had their tumors irradiated with a 3 W/cm² 1064 nm continuous wave NIR laser (IPG Photonics) for 30 seconds. After treatment, changes in tumor volume for all mice were monitored every three days for 45 days by digital caliper measurements. Mice were removed from the study (considered “dead”) when their tumor volumes exceeded 1000 mm³ or were deemed moribund by veterinary consult.

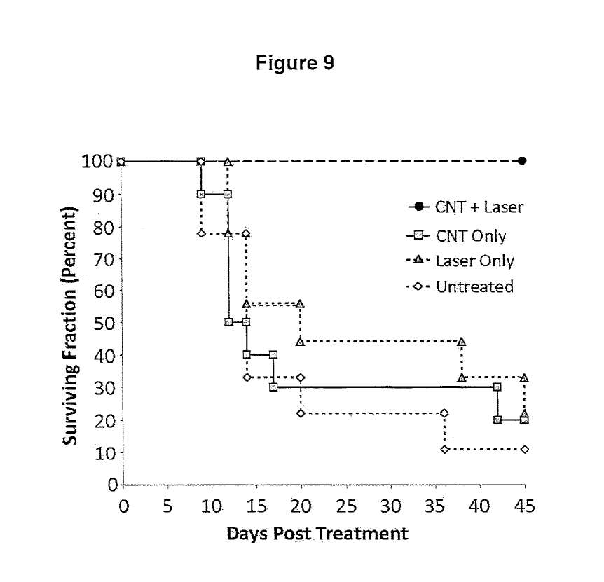

As shown in the Kaplan-Meier analysis of FIG. 9, control group animals displayed marked tumor burdens (Untreated=1125.7±146.7 mm³; Laser Only=1020.9±209.1 mm³; CNT Only=1113.8±205.9 mm³; mean*standard error) and significant mortality (Untreated=11% alive; Laser Only=22% alive; CNT Only=20% alive) 45 days post treatment. In contrast, NMTT led to complete tumor regression (CNT+Laser=0±0 mm³) and significantly enhanced overall survival (100%) relative to the control groups (P<0.05). For the Kaplan-Meier plot nonparametric survival analysis, models were fit to compare groups. Log-rank tests were used to determine differences between groups.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

That which is claimed is:

1. A method of treating cancerous tissue comprising:
inducing cell death in cancer stem cells in vivo during hyperthermic treatment of the tissue, inducing cell death comprising:
positioning nanoparticles adjacent to the cancer stem cells, the nanoparticles having an aspect ratio of 10 to 1,000; and
irradiating the nanoparticles with infrared radiation to heat the nanoparticles, thereby resulting in membrane damage and death of the cancer stem cells.

2. The method of claim 1, wherein the membrane damage comprises membrane permeabilization.

3. The method of claim 1 further comprising inducing death in bulk cancer cells of the cancerous tissue.

4. The method of claim 3, wherein the death of the bulk cancer cells is necrotic cell death, apoptotic cell death or a combination thereof.

5. The method of claim 3, wherein the cancer stem cells are not enriched in the cancerous tissue during the hyperthermic treatment of the tissue.

6. The method of claim 1, wherein the cancerous tissue is breast tissue, and the cancer stem cells are breast cancer stem cells.

7. The method of claim 1, wherein the nanoparticles comprise organic nanoparticles, inorganic nanoparticles or mixtures thereof.

8. The method of claim 7, wherein the organic nanoparticles comprise single-walled carbon nanotubes, multi-walled carbon nanotubes or mixtures thereof.

9. The method of claim 7, wherein the inorganic nanoparticles comprise metal nanoparticles, metal oxide nanoparticles or mixtures thereof.

10. The method of claim 1, wherein the proliferative ability of cancer stem cells of the tissue not undergoing necrotic cell death or apoptotic cell death subsequent to irradiation of the nanoparticles is diminished or abrogated.

11. The method of claim 1, wherein the nanoparticles directly contact the cancer stem cells, or are positioned about 1000 nm or less from the cancer stem cells.

12. The method of claim 1, wherein the nanoparticles are present in an amount of 10 μg/ml to 500 μg/ml.

13. The method of claim 1, wherein inducing cell death consists essentially of:
positioning nanoparticles adjacent to the cancer stem cells, the nanoparticles having an aspect 10-1,000; and
irradiating the nanoparticles with infrared radiation to heat the nanoparticles, thereby resulting in membrane damage to the cancer stem cells.

14. A method of treating cancerous tissue comprising:
diminishing or abrogating the proliferative ability of cancer stem cells of the tissue in vivo, diminishing or abrogating the proliferative ability comprising:
positioning nanoparticles in the tissue; and
heating the nanoparticles having an aspect ratio of 10 to 1,000 by irradiating the nanoparticles with infrared radiation to heat the tissue, the cancer stem cells surviving the heating.

15. The method of claim 14, wherein bulk cancer cells of the cancerous tissue are killed by the heating of the tissue.

16. The method of claim 15, wherein killing the bulk cancer cells comprises inducing necrotic cell death, apoptotic cell death or a combination thereof.

17. The method of claim 14, wherein the cancerous tissue is breast tissue, and the cancer stem cells are breast cancer stem cells.

18. The method of claim 14, wherein the nanoparticles comprise single-walled carbon nanotubes, multi-walled carbon nanotubes or mixtures thereof.

19. The method of claim 14, wherein the nanoparticles comprise metal nanoparticles, metal oxide nanoparticles or mixtures thereof.

20. The method of claim 14, wherein diminishing or abrogating the proliferative ability consists essentially of:
positioning nanoparticles in the tissue; and
heating the nanoparticles having an aspect 10 to 1,000 by irradiating the nanoparticles with infrared radiation to heat the tissue, the cancer stem cells surviving the heating.
